(19) United States
(12) Patent Application Publication
(10) Pub. No.: US 2024/0026274 A1
(43) Pub. Date: Jan. 25, 2024

(54) APPARATUS, SYSTEM AND METHOD FOR THERMAL FOAM DETECTION

(71) Applicant: GLOBAL LIFE SCIENCES SOLUTIONS USA LLC, Marlborough, MA (US)

(72) Inventors: JEFFREY SOCHA, BOYLSTON, MA (US); ALAN STOCKDALE, PROVIDENCE, RI (US); DANIEL STELLA, BOLTON, MA (US)

(73) Assignee: GLOBAL LIFE SCIENCES SOLUTIONS USA LLC, MARLBOROUGH, MA (US)

(21) Appl. No.: 17/871,573

(22) Filed: Jul. 22, 2022

(51) Int. Cl. C12M 1/21; H04N 5/33; G01V 8/00; C12M 1/34; C12M 1/00

(52) U.S. Cl. CPC C12M 41/02; H04N 5/33; G01V 8/005; C12M 41/12; C12M 23/22; C12M 23/14

(57) ABSTRACT

A foam identification system including a thermal imaging camera and a controller connected to the thermal imaging camera. The thermal imaging camera images a surface of a liquid in a vessel that is exposed to a headspace of the vessel. The headspace being either warmer or cooler than the liquid. The camera and the controller detect a change in temperature of the exposed surface of the liquid to identify foam on the exposed surface of the liquid.

### BACKGROUND

### Technical Field

Embodiments of the invention relate generally to bioprocessing apparatus, systems, and methods, and more particularly, to thermally observing and analyzing a fluid in a bioreactor to detect foam.

### Discussion of Art

Bioreactors are often employed to carry out biochemical and/or biological processes and/or manipulate liquids and other products of such processes. Such bioreactors often include flexible or collapsible single-use disposable bags that are supported by an outer rigid structure such as a stainless-steel shell or frame. The bags are made of thin flexible sheets of plastic film and are positioned within the rigid shell and filled with the desired fluid for processing.

Growing biological materials such as mammalian cells, bacteria or yeast in a bioreactor often results in the production of an unwanted foam layer which floats at the top of the fluid in the bioreactor, e.g., in a headspace of a bioreactor bag. This foam layer is the result of several factors including the addition of pressurized air to sustain aerobic microorganisms, nutrients and growth factors present in the liquid growth media, and waste products generated by the microorganisms. Over time, this foam layer may become unacceptably thick, and, if untreated, could potentially foul the exhaust port and filter of a bioreactor, prevent CO₂ from escaping, and negatively affect the structural integrity of the bag. Foam also forms a barrier to liquids injected from above the fluid in the bioreactor and is problematic even at low fluid volume levels.

To reduce the foam layer to a reasonable thickness, chemical solutions such as antifoam compounds are typically employed. With respect to such compounds, several applications may be needed during a single production run to ensure effectiveness. Conversely, too much antifoam compound can be detrimental to the biological materials in the reactor. Mechanical solutions, such as thermal probes and foam breakers also exist, however, they are more effective at reducing substantial amounts of existing foam rather than inhibiting foam formation.

In view of the above, accurate detection and monitoring of foam in a bioreactor bag is important to determine when intervention is necessary. While foam detection solutions exist, they only detect foam levels in a small area or, in some instances, at a single point in the bioreactor bag, rather than-assessing the entirety of the exposed fluid surface in the bag. Moreover, many such systems have been found to be generally effective only for the detection of extreme foam events in which the biological materials, or the structure of bag itself, may already be compromised. Known solutions are also relatively large and expensive and do not function to ensure that, for example, the requisite amount of antifoam compound is applied in response to actual foam levels in a bag and do not have the capability to quantify the amount of foam present.

In view of the above, there is a need for an apparatus and system for observing a fluid in a bioreactor bag that provides for improved detection, monitoring, and mitigation of foam in the bag.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

In an embodiment, a foam identification system includes a thermal imaging camera and a controller connected to the thermal imaging camera. The thermal imaging camera images a surface of a liquid in a vessel that is exposed to a headspace of the vessel, the headspace being either warmer or cooler than the liquid. The camera and the controller detect a change in temperature of the exposed surface of the liquid to identify foam on the exposed surface of the liquid.

In another embodiment of the invention, a method for identifying foam on a surface of a liquid in a vessel includes obtaining a temperature measurement of a liquid in the vessel, determining whether a headspace of the vessel is warmer or cooler than the liquid, obtaining one or more temperature measurements of a surface of the liquid exposed to the headspace of the vessel via a thermal imaging camera, detecting a change in temperature of the exposed surface of the liquid, and identifying foam on the exposed surface based on the detected change in temperature.

In yet another embodiment, a bioreactor system includes a frame, a thermal imaging camera, and a controller connected to the thermal imaging camera. The frame houses and supports a vessel. The thermal imaging camera is secured to the frame and images a surface of a liquid exposed to a headspace of the vessel. The headspace being either warmer or cooler than the liquid. The camera and the controller detect a change in temperature of the exposed surface of the liquid to identify foam on the exposed surface.

### DRAWINGS

The present invention will be better understood from reading the following description of non-limiting embodiments, with reference to the attached drawings, wherein below:

FIG. 1 is a front elevational view of a bioreactor system suitable for use with a foam identification system, according to an embodiment of the present invention.

FIG. 2 is a diagram of a foam identification system, according to an embodiment of the present invention.

FIG. 3 is diagram comparing temperatures within a bioreactor system over time.

FIG. 4 is a top view of a diagram of foam magnitude within a bioreactor system as determined by a foam identification system in accordance with an embodiment of the present invention.

FIG. 5 is a side sectional view of a portion of a wall of a vessel configured for use with embodiments of the present invention.

FIG. 6 is a side sectional view of a view port of a vessel configured for use with embodiments of the present invention.

FIG. 7 is an isometric view of an air curtain configured for use with embodiments of the present invention.

FIG. 8 is a diagram of a foam identification system incorporating an air curtain according to an embodiment of the present invention.

FIG. 9 is a side view of a foam identification system according to an alternative embodiment of the present invention.

FIG. 10 is a top view of a foam identification system according to an alternative embodiment of the present invention.

FIG. 11 is a side view of the foam identification system of FIG. 10.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, or a rigid container, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, fermentation systems, mixing systems, media/buffer preparation systems, and filtration/purification systems.

As used herein, the term “bag” means a flexible or semi-rigid container or vessel used, for example, as a bioreactor or mixer for the contents within. While embodiments of the present invention are described as for use with bioprocessing bags, including but not limited to bioreactor bags and mixer bags, embodiments may also be configured for use with other bags or vessels. Similarly, embodiments may be used to image, assess, and mitigate/treat other characteristics or conditions, in addition to the accumulation of foam in a bioreactor.

Further, while embodiments are described in connection with single use, stirred tank bioreactors and bioreactor systems, they are not limited to the same and may be used with a variety of vessels and associated equipment used in biological or biochemical processing. Additionally, embodiments may be suitable for use in identifying foam in other non-biological/biochemical contexts. Certain embodiments may be useful in detecting other non-foam related conditions or events on a surface that may be identified via a temperature difference or change as described herein.

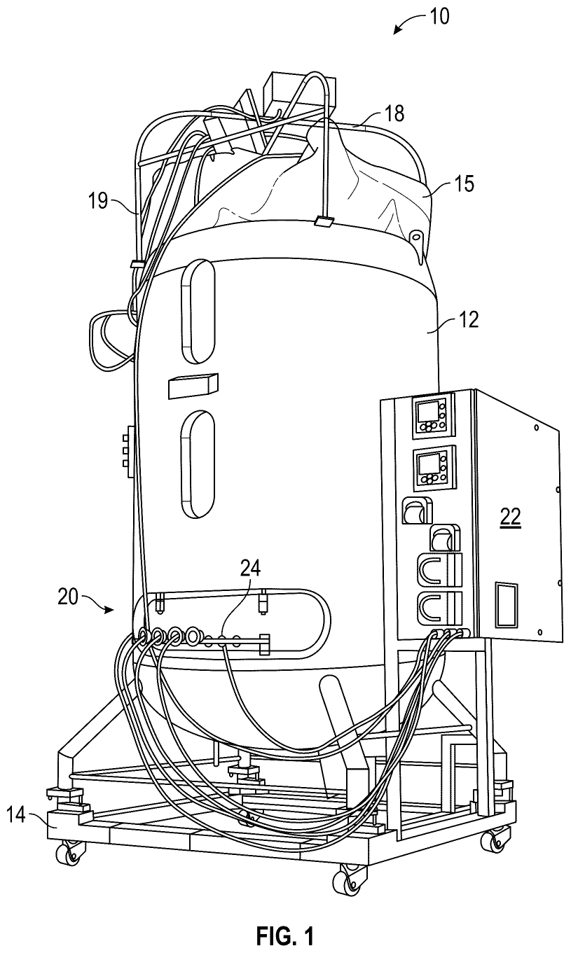

With reference to FIG. 1, a bioreactor system 10 suitable for use with embodiments of the invention is illustrated. The bioreactor system 10 includes a generally rigid bioreactor housing 12 mounted atop a frame 14. The rigid housing 12 may be formed, for example, from stainless steel, polymers, composites, glass, or other metals, and may be cylindrical in shape, although other shapes may also be utilized without departing from the broader aspects of the invention. As will be appreciated, the housing is configured to house and support a vessel, e.g., bioreactor bag 15. In certain embodiments, the housing 12 may be a substantially rectangular mixer housing.

As shown, a single-use, flexible bioreactor bag 15 is disposed within the housing 12. As mentioned, the housing 12 can be any size (or shape) as long as it is capable of supporting a vessel such as a single-use flexible bioprocess bag 15. For example, according to one embodiment, the housing 12 is capable of accepting and supporting a 10-2000 L flexible or collapsible bioprocess bag.

The bioreactor system 10 further includes a support structure 18 to which various equipment utilized in biochemical and/or biological processes are attached. The support structure 18 may also be used to lift and hold the bag 15 in place within the housing 12. The support structure 18 is shown as having a plurality of leg portions 19, but other configurations may be employed.

The housing 12 includes an opening or aperture 20 where, among other things, a temperature probe 24 can be inserted into a thermowell or port in the vessel 15 and then be coupled via e.g., a cable, to the instrument tower 22. As will be appreciated, the temperature probe 24 provides a temperature of a fluid in the vessel 15.

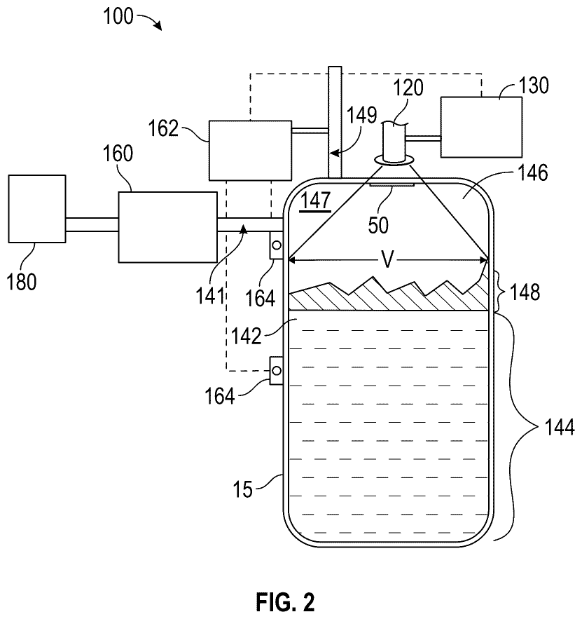

Referring now to FIG. 2, a foam identification system 100 according to an embodiment of the invention is depicted. As shown, the foam identification system 100 includes a thermal imaging camera 120 and a controller 130 operatively connected to the thermal imaging camera 120. In embodiments, the thermal imaging camera 120 is secured to the support structure 18 of the frame 14 (FIG. 1). As described in greater detail below, the thermal imaging camera 120 images a surface 142 of a liquid 144 in the vessel (e.g., bag) 15. In particular, the camera 120 images a surface 142 that is exposed to a headspace 146 of the vessel 15. The headspace 146 being the volume within the vessel 140 not occupied by liquid 144 or foam 148. The headspace 146 includes a gas 147, e.g., air, retained within the vessel 15, which is in contact with the exposed surface 142 of the liquid 144.

In the depicted embodiment, the vessel 15 includes an inlet 141 and an outlet 149 that allow passage of fluids, e.g., the gas 147, into and out of the headspace 146. The gas 147 within the headspace 146 is either warmer or cooler than the liquid 144 in the vessel 15. The temperature of ambient air surrounding the vessel has a large influence on the temperature of the gas 147 within the headspace 146. In embodiments, there is a sufficient temperature different between the temperature of the liquid 144 and the temperature of the gas 147 (e.g., 2-4° C.), even when the temperature of the gas 147 flowing into the headspace 146 through the inlet 141 is not controlled. Given the temperature difference between the temperature of the gas 147 and the temperature of the liquid 144, the camera 120 and the controller 130 can detect a change in temperature of the exposed surface 142 to identify the presence and quantity of foam 148 on the surface 142.

In embodiments, the thermal imaging camera 120 utilizes mid-wavelength to far-wavelength infrared imaging to collect temperature and radiometric data by reading heat directly without requiring illumination. The mid-wavelength and far-wavelength infrared imaging discussed herein is not to be confused with shortwave or near-wavelength infrared imaging, which requires illumination to generate the image. In some embodiments, the thermal imaging camera 120 detects infrared light having a wavelength of from about 7 μm to about 14 μm.

Significantly, the thermal imaging camera 120 sees a wide field of view V as opposed to a point source, which is important due to the unpredictable nature of foam buildup. In embodiments, the field of view V is substantially the entirety of the exposed surface 142. In certain embodiments, the thermal camera 120 may utilize a wide-angle lens and may include auto-focus functionality.

In an embodiment, the camera 120 and the controller 130 detect a change in temperature of the exposed surface 142 of the liquid 144 that is indicative of the formation or presence of foam 148 on the exposed surface 142. More specifically, the temperature of the exposed surface 142 of the liquid 144 is going to depart, i.e., increase or decrease, from the temperature of the liquid 144 in the vessel 15 that is not exposed to the head space, depending on whether the head space is warmer or cooler than the liquid 144.

In certain embodiments, thermal imaging camera 120 is radiometric. A thermal imaging camera 120 with an integrated radiometer may provide temperature data for every pixel of the image enabling the system 100 to tabulate and quantify the foam (e.g., create a histogram, etc.) by percentage area, height, persistence, etc. The thermal imaging camera 120 allows the system 100 to obtain a heat-map without the need for an algorithm and model the heat-map as height and/or over time.

The foam identification system 100 may also include a temperature control system that maintains the headspace 146 at a temperature warmer or cooler than a temperature of the liquid 144. The temperature control system may include a gas temperature controller 160 and at least one temperature monitor 162. In a specific embodiment, the temperature control system may utilize one or more temperature monitors 162 that may include a monitor 162 for gas flowing into the vessel 15 via the inlet 141, a monitor 162 for temperature of the headspace 146, and/or a monitor for gas flowing out of the vessel outlet 149. As will be appreciated, a variety of temperature monitors may be utilized, e.g., temperature probe sensors and the like, without departing from the scope of the invention. In embodiments, temperature sensors 164 are employed to monitor areas of Interest, i.e., the exterior surface of the vessel 15, without making contact with the environment inside the vessel. The gas temperature controller 160 and/or the temperature monitor 162 communicate with the controller 130 to provide the desired temperature.

The temperature monitors 162 detect the temperature of gas flowing into the headspace 146 through the inlet 141, the temperature of gas flowing out of the headspace 146 through the outlet 149, and/or the temperature of the gas 147 within the headspace 146. In response to measured temperatures, the temperature control system may adjust the head space temperature to maintain it at a certain set temperature or a number of degrees (e.g., 5° C.) higher or lower than the temperature of the liquid 144 in the vessel 15. In embodiments, the gas temperature controller 160 may be provided with a controlled temperature value of the liquid 144 in the vessel 15, may be provided with an ambient air value of the air outside the vessel 15, may be operatively connected to the temperature probe 24 in the thermowell or port in the vessel 15 and/or the instrument tower 22, or may be operatively connected to the temperature monitor 162 detecting the temperature of gas flowing into the headspace 146 through the inlet 141.

In other embodiments, the temperature of the liquid 144 in the vessel 15 may be approximated by measuring the temperature of the vessel wall through the exposed surface. More specifically, the temperature of the vessel wall through the exposed surface (if unobstructed by foam) may be a suitable calibration proxy for the controlled vessel temperature. That is, if, for example, the controlled vessel temperature is 37° C., the thermal camera may be able to record this same temperature value by thermal imaging the vessel wall at or through the exposed surface. In this manner, the system may continuously calibrate without having to be provided with real-time temperate measurements of the liquid 144.

As will be appreciated, the gas temperature controller 160 may heat or cool the gas from the mass flow controller 180 prior to it venting into the headspace 146 via inlet 141. In embodiments, the gas temperature controller 160 need not actively heat or cool the gas but may simply contain a coil of tubing that approaches the ambient air temperature. Such embodiments may be effective where the difference between the temperature of the liquid 144 and that of the ambient air outside of the vessel 15 is sufficient for foam detection. For example, an ambient air temperature of ˜22° C. may be sufficient.

In certain embodiments, a temperature control system and/or gas introduction may not be necessary and may be entirely omitted. Here, the gas 147 in the headspace 146 will be sufficiently cooler than the liquid 144 due to the top of the vessel (e.g., bag) 15 that contains the headspace 146 protruding above and outside of the rigid (e.g., stainless steel) vessel housing 12.

Alternative means of controlling the temperature of the gas 147 within the headspace 146 (e.g., heater, lamps, ambient air, etc.) do not depart from the invention disclosed herein.

Referring now to FIGS. 2-4, in embodiments, the foam identification system 100 determines the presence and/or magnitude of foam for substantially an entirety of the surface 142 of the liquid 144 exposed to the headspace 146. Typically, the headspace 146 will have a lower temperature than that of the liquid in the vessel 15. The headspace 146 is exposed to ambient room air, which, at about 22° C., is generally substantially cooler than the liquid temperature, which is often set at about 37° C. Foam 148 on the exposed surface 146 will approach the head space 146 temperature over time. Liquid on the exposed surface is less influenced by head space 146 temperature and will remain relatively close to the controlled vessel temperature. As such, the presence and/or magnitude of foam may be identified based on temperature changes detected via thermal imaging, radiometric data, and/or the rate at which the exposed surface 142 changes temperature.

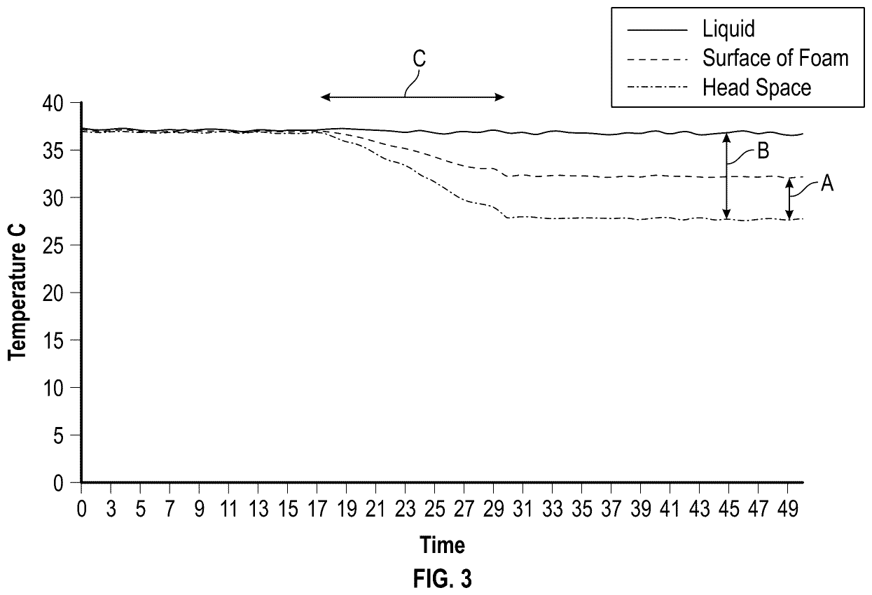

As shown in FIG. 3, as the temperature of the gas 147 within the headspace 146 decreases over time (represented by the lowest line on the chart), the foam temperature (middle line) approaches the headspace temperature, as denoted by reference “A.” As mentioned above, the liquid 144 (top line) is less influenced by the temperature of the headspace 146, as denoted by reference “B.” The rate of the surface temperature change as the gas 147 within the headspace 146 changes, as denoted by reference “C,” is also an indicator of the formation and magnitude of foam on the surface 142. As will be appreciated, a relatively fast rate of temperature change is indicative of the formation of a relatively high amount/magnitude of foam. Conversely a low rate of change is indicative of a relatively low amount/magnitude of foam. Very low rates of change may be indicative of normal cooling of the exposed surface 142 without the formation of foam.

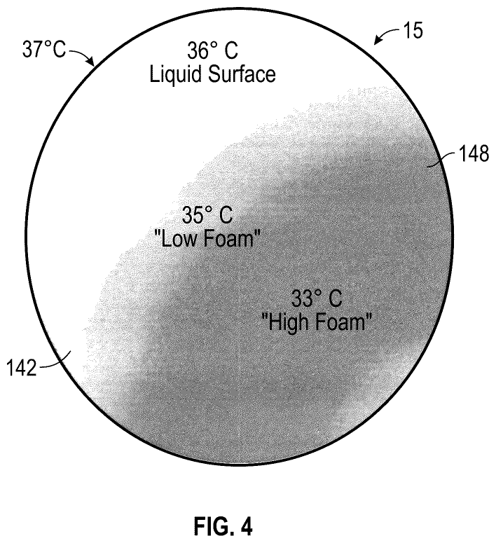

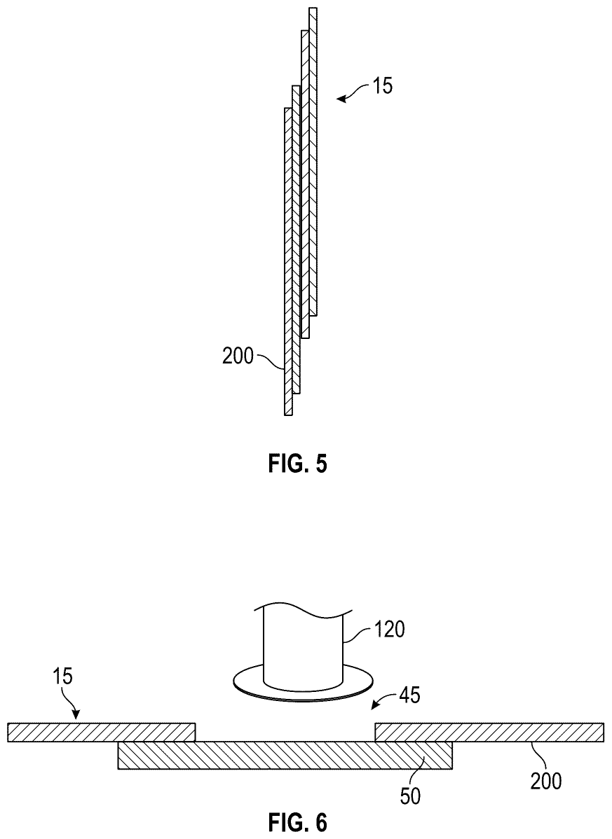

By way of non-limiting example, FIG. 4 illustrates a vessel 15 that is set at a controlled vessel temperature of 37° C., with a head space gas 147 that has a lower temperature than the liquid 144 temperature. In certain embodiments, it has been found that a temperature difference (plus or minus) of at least 0.5° C. between the head space gas 147 and the liquid 144 temperature is sufficient for foam identification, although smaller differences in temperature may also be sufficient. The exposed surface 142 measures 36° C., and the presence of a low amount/magnitude of foam is indicated by a measured surface temperature of 35° C., or 0.5 to 1.5 degrees lower than the exposed surface 142 temperature. A high amount/magnitude of foam is indicated by an exposed surface 142 temperature of 33° C., or 2-3 degrees lower than the liquid 144 temperature. In this example, the vessel wall temperature measured through a foamless portion of the exposed surface 142 was found to be 37° C., the same as the controlled vessel temperature.

As will be appreciated, the specific temperatures and/or the size of the difference in temperature (higher or lower) indicative of foam may vary based on several factors including, but not limited to, the temperature of the head space gas 147 and liquid 144, and the head-sweep gas flow temperature and rate.

As will be appreciated, embodiments of the invention are useful in determining when chemical or mechanical defoaming should occur, the amount of defoaming required given the magnitude/rate of formation of foam, and the efficacy of defoaming treatments.

Referring now to FIGS. 2 and 6, in embodiments, the foam identification system 100 includes a vessel 15 having a view port 50 that allows the thermal imaging camera 120 to image the exposed surface 142 of the liquid 144. In some embodiments, the view port 50 may be heated to reduce condensation or may be equipped with an air curtain, as described in greater detail below.

In embodiments, the vessel 15 has a multi-layer film construction that includes an innermost layer of wetted material 200 (e.g., Polyethylene) that is in contact with the liquid in the vessel. The view port 50 may be formed on or bonded to the wetted material 200. In certain embodiments, the view port 50 is made from a low-density polyethylene (LDPE), a material which has been found to have excellent transmissivity in the spectral range of interest, e.g., 7-14 μm. As will be appreciated, the thickness of the view port 50 may vary depending on material properties. Other materials having the requisite transmissivity may be utilized without departing from the scope of the invention. In certain embodiments, polypropylene and polystyrene may be utilized.

In certain embodiments, the view port 50 may be the same single or multi-layer material as the material the vessel 15 itself. In other words, the vessel 15 may not have a dedicated view port having a construction departing from that of the vessel 15. For example, 15 to 20 mil thick LDPE sheeting may provide suitable transmissivity and structure for such embodiments. In yet other embodiments, the port 50 may be the inside wetted material 200 layer and may be formed by simply removing the layers that lie on top of the wetted material 200.

In certain embodiments, the view port 50 is round and is substantially wider/larger in diameter than the lens of the thermal camera 120. Other view port 50 sizes and shapes may be employed without departing from the invention.

As mentioned, the camera 120 may mounted on the support structure 18 such that it is positioned above the vessel 15 and aimed vertically downward such that substantially an entirety of the exposed surface 142 may be imaged. In this regard, the view port 50 may be located on an upper or top surface of the vessel 15. As will be appreciated, the view port 50 may be in a variety of locations, as long as substantially an entirety of the exposed surface 142 may be imaged.

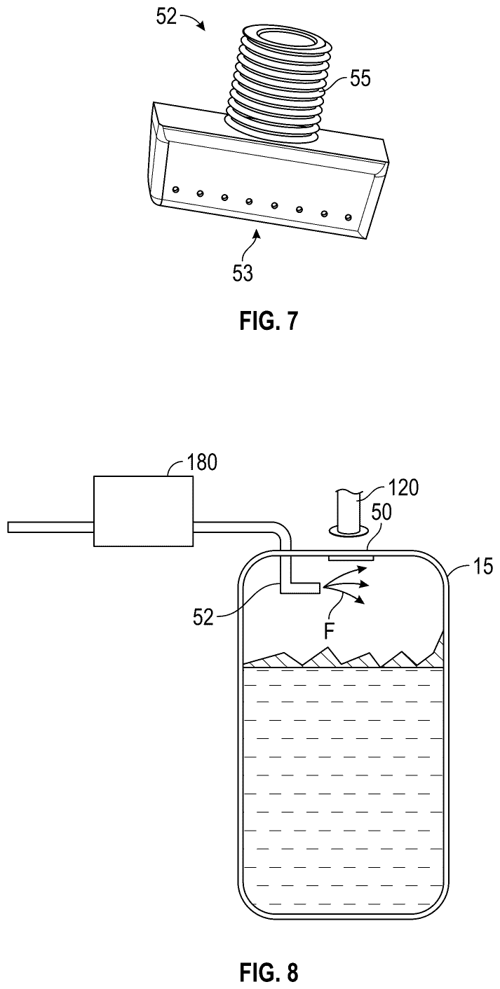

In one embodiment, the foam identification system 100 includes an air curtain 52, as depicted in FIGS. 7 and 8. The air curtain 52 reduces vessel condensation to facilitate thermal imaging of the exposed surface 142. In embodiments, the air curtain 52 is located within the vessel (e.g., bag) 15 and is aimed at the view port 50, or other optically clear view area of the vessel 15. The air curtain 52 may be removable or fixedly attached to a wall of the vessel 15 and may utilize gas (e.g., air, O₂ or N₂) from existing head-sweep gas flow from the mass flow controller 180. As will be appreciated, in embodiments where the air curtain 52 utilizes existing gas flow, no additional hardware is required, only the addition of the air curtain 52 to the vessel 15. Moreover, existing head-sweep gas flow provides a supply of gas that has a very low dew point of less than −40° C., ideal for condensation prevention. Moreover, use of the air curtain 52 may render the use of a heater unnecessary.

Referring specifically to FIG. 7, an exemplary air curtain 52 includes a nozzle or outlet portion 53 through which gas/air flow is directed and a threaded base portion 55. The threaded base portion 55 may be directly attached to or otherwise in fluid communication with the mass flow controller 180 (FIG. 8).

In use, the air curtain 52 directs a flow of gas/air F toward the view port 50 to clear the area of condensation. In certain embodiments, the air curtain 52 may be selectively positionable so that the flow of gas F can be directed by an operator to maximize condensation removal. Moreover, the velocity of the flow of gas/air F may be varied depending upon the moisture content of the air in the head space, air temperature in the head space, or other variables. In this regard, the air curtain 52 may be paired with a sensor or meter to measure moisture content and the like.

As will be appreciated, the air curtain 52 may be used to clear a portion of a vessel/bag of condensation for purposes other than thermal imaging, e.g., for various external optical measurements.

In certain other embodiments, an air knife may be employed, though additional pressurized air and flow control may be required in such configurations.

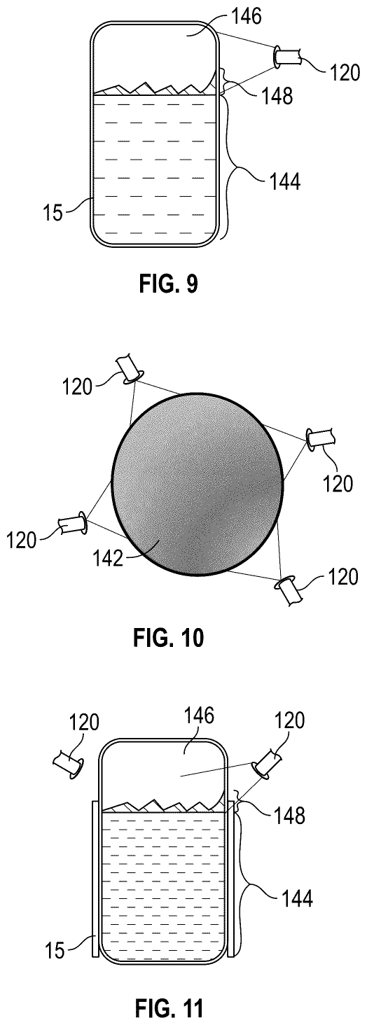

Referring now to FIGS. 9-11, alternative arrangements of thermal cameras may be employed. In one embodiment, the thermal camera 120 may be located such that it images a side of the headspace of the vessel 15. Such an arrangement may be suitable for vessels (e.g., bags) that are entirely made from a material that is substantially transparent in the thermal spectral range, or vessels with a side view port.

In another embodiment, the system may include a plurality of thermal cameras 120 spaced about the periphery of the of the vessel 15 and aimed at the headspace. In certain embodiments, one or more thermal cameras may be built into the rigid bioreactor housing 12. In yet other embodiments, a thermal camera may be integral to the vessel/bag itself. In embodiments, with multiple cameras or cameras built into the vessel/bag, lower resolution thermal imaging cameras may be employed to reduce cost.

In use, the system 100 identifies the presence of and/or a magnitude of foam 148 on the surface 142 of the liquid 144 exposed to the headspace 146 in a number of ways. In one embodiment, the system 100 detects a rate of temperature change on the exposed surface 142. In another embodiment, the thermal camera 120 and controller 130 identify areas of foam 148 by comparing a temperature of the exposed surface 142 to a temperature of the liquid 144 to assess whether the exposed surface 142 is a warmer or cooler than the liquid 144 by a predetermined amount that is indicative of foam.

In yet another embodiment, the thermal camera 120 and controller 130 identify areas of the exposed surface 142 as foam 148 by comparing a temperature of the exposed surface 142 to a temperature of the headspace 146 to assess whether the exposed surface 142 is within a predetermined temperature range of the headspace temperature that is indicative of foam.

A method of identifying foam 148 on a surface of a liquid 144 in a vessel 15 is provided. The method includes obtaining a temperature measurement of a liquid 144 in the vessel 15, determining whether a headspace 146 of the vessel 15 is warmer or cooler than the liquid 144, obtaining one or more temperature measurements of a surface 142 of the liquid 144 exposed to the headspace 146 of the vessel 15 via a thermal imaging camera 120, detecting a change in temperature of the exposed surface 142 of the liquid 144, and identifying foam 148 on the exposed surface 142 based on the detected change in temperature.

In one embodiment, the step of identifying foam 148 on the exposed surface 142 includes comparing a temperature of the exposed surface 142 of the liquid 144 to a temperature of the liquid 144 to assess whether the exposed surface 142 is a warmer or cooler than the liquid 144 in a predetermined amount that is indicative of foam. In another embodiment, the step of identifying foam 148 on the exposed surface 142 includes comparing a temperature of the exposed surface 142 to a temperature of the headspace 146 to assess whether the exposed surface 142 is within a predetermined temperature range of the headspace temperature that is indicative of foam.

In yet another embodiment, the step of identifying foam 148 on the exposed surface 142 includes obtaining a plurality of temperature measurements of the exposed surface 142 of the liquid 144, determining a rate of temperature change on the exposed surface 142 from the plurality of temperature measurements of the exposed surface 142, and identifying the presence and/or a magnitude of foam 148 on the surface of the liquid 144 by comparing the rate of temperature change to a predetermined value that is indicative of foam.

In one embodiment, the step of identifying foam 148 incorporates maintaining a temperature of the headspace 146 to be either warmer or cooler than the liquid 144. In one embodiment, the method of identifying foam 148 also includes mitigating detected foam 148 on the exposed surface 142 of the liquid 144, for example, by application of an antifoaming agent into the vessel 15.

In embodiments, the method of identifying foam 148 also includes removing condensation from the view port 50 of vessel 15 via a condensation prevention system (e.g., the air curtain 52, etc.) to facilitate identification of foam 148 by the thermal imaging camera 120.

In some embodiments, the foam identification system 100 provides defoaming injection feedback, by analyzing the foam magnitude during and after use of mechanical or gaseous solutions in addition to chemical anti-foam agents. The thermal imaging camera 120 provides data that quantifies an input of anti-foam agents and/or a response of the foam 148 to the anti-foam agents.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A foam identification system comprising:
a thermal imaging camera configured to image a surface of a liquid and a foam in a vessel that is exposed to a headspace of the vessel, the headspace being either warmer or cooler than the liquid and the foam;
a controller operatively connected to the thermal imaging camera; and
wherein the camera and controller are configured to detect a change in temperature of the exposed surface to identify foam on the exposed surface.

2. The foam identification system of claim 1, further comprising:
a temperature control system configured to maintain the headspace at a temperature warmer or cooler than the liquid.

3. The foam identification system of claim 2, wherein the temperature control system comprises:
a gas temperature controller and at least one temperature monitor to detect the temperature of at least one of gas flowing into the headspace, gas flowing out of the headspace, and gas surrounding the vessel.

4. The foam identification system of claim 1, further comprising:
a temperature monitor configured to measure the temperature of the headspace.

5. The foam identification system of claim 1, wherein the thermal imaging camera provides data that quantifies an input of anti-foam agents and/or a response of the foam to the anti-foam agents.

6. The foam identification system of claim 1, wherein the thermal imaging camera detects infrared light having a wavelength of from about 7 μm to about 14 μm.

7. The foam identification system of claim 1, wherein the thermal imaging camera is radiometric.

8. The foam identification system of claim 1, further comprising:
a vessel having a view port configured to allow the thermal imaging camera to image the exposed surface of the liquid.

9. The foam identification system of claim 8, wherein the view port is heated to reduce condensation.

10. The foam identification system of claim 1, further comprising:
an air curtain configured to reduce vessel condensation to facilitate thermal imaging of the exposed surface.

11. The foam identification system of claim 1, further comprising:
a housing configured to house and support the vessel, the housing having a support structure to which the thermal imaging camera is mounted.

12. The foam identification system of claim 11, wherein the camera is mounted on the support structure so that it is positioned above the vessel and aimed vertically downward so that substantially an entirety of the exposed surface may be imaged.

13. The foam identification system of claim 1, wherein the vessel is a collapsible bioreactor bag.

14. The foam identification system of claim 1, wherein the system is configured to identify the presence of and/or a magnitude of foam on the surface of the liquid exposed to the headspace by detecting a rate of temperature change on the exposed surface.

15. The foam identification system of claim 1, wherein the camera and controller identify areas of the exposed surface by comparing a temperature of the exposed surface to a temperature of the liquid to assess whether the exposed surface is a warmer or cooler than the liquid in a predetermined amount that is indicative of foam.

16. The foam identification system of claim 1, wherein the camera and controller identify areas of the exposed surface as foam by comparing a temperature of the exposed surface to a temperature of the headspace to assess whether the exposed surface is within a predetermined temperature range of the headspace temperature that is indicative of foam.

17. The foam identification system of claim 1, wherein the headspace is cooler than the liquid in the vessel.

18. A method of identifying foam on a surface of a liquid in a vessel comprising the steps of:
obtaining a temperature measurement of a liquid in the vessel;
determining whether a headspace of the vessel is warmer or cooler than the liquid;
obtaining at least one temperature measurement of a surface of the liquid and of a foam exposed to the headspace of the vessel via a thermal imaging camera;
detecting a change in temperature of the exposed surface; and
identifying foam on the exposed surface based on the detected change in temperature.

19. The method of claim 18, wherein the step of identifying foam on the exposed surface comprises:
comparing a temperature of the exposed surface to a temperature of the liquid to assess whether the exposed surface is a warmer or cooler than the liquid in a predetermined amount that is indicative of foam.

20. The method of claim 18, wherein the step of identifying foam on the exposed surface comprises:
comparing a temperature of the exposed surface to a temperature of the headspace to assess whether the exposed surface is within a predetermined temperature range of the headspace temperature that is indicative of foam.

21. The method of claim 18, wherein the step of identifying foam on the exposed surface comprises:
obtaining a plurality of temperature measurements of the exposed surface;
determining a rate of temperature change on the exposed surface from the plurality of temperature measurements of the exposed surface; and
identifying the presence and/or a magnitude of foam on the surface of the liquid by comparing the rate of temperature change to a predetermined value that is indicative of foam.

22. The method of claim 18, further comprising providing data that quantifies an input of anti-foam agents and/or a response of the foam to the anti-foam agents.

23. The method of claim 18, further comprising the step of:
maintaining a temperature of the headspace to be either warmer or cooler than the liquid.

24. The method of claim 18, wherein the headspace is cooler than the liquid.

25. The method of claim 18, further comprising the step of:
mitigating detected foam on the exposed surface of the liquid.

26. The method of claim 18, wherein the thermal imaging camera detects infrared light having a wavelength of from about 7 μm to about 14 μm.

27. The method of claim 18, wherein the thermal imaging camera is radiometric.

28. The method of claim 18, wherein the step of determining whether a headspace of the vessel is warmer or cooler than the liquid comprises:
obtaining a temperature of the headspace via a headspace temperature sensor and comparing the headspace temperature to the temperature measurement of the liquid.

29. The method of claim 18, wherein the step of determining whether a headspace of the vessel is warmer or cooler than the liquid comprises:
obtaining a temperature a portion of the vessel wall in the headspace via the thermal imaging camera and then comparing the vessel wall temperature to the temperature measurement of the liquid.

30. The method of claim 18, further comprising the step of:
removing condensation from the vessel via a condensation prevention system to facilitate identification of foam by the thermal imaging camera.

31. A bioreactor system comprising:
a housing configured to house and support a vessel;
a thermal imaging camera secured to the housing, the thermal imaging camera configured to image a surface of a liquid and a foam exposed to a headspace of the vessel, the headspace being either warmer or cooler than the liquid;
a controller operatively connected to the thermal imaging camera; and
wherein the camera and controller are configured to detect a change in temperature of the exposed surface to identify foam on the exposed surface.

32. The bioreactor system of claim 31, further comprising:
a temperature control system configured to maintain the headspace at a temperature warmer or cooler than the liquid.

33. The bioreactor system of claim 32, wherein the temperature control system comprises:
a gas temperature controller and at least one temperature monitor to detect the temperature of at least one of gas flowing into the headspace, gas flowing out of the headspace, and gas surrounding the vessel.

34. The bioreactor system of claim 31, further comprising:
a temperature monitor configured to measure the temperature of the headspace.

35. The foam identification system of claim 31, wherein the thermal imaging camera provides data that quantifies an input of anti-foam agents and/or a response of the foam to the anti-foam agents.

36. The bioreactor system of claim 31, wherein the thermal imaging camera detects infrared light having a wavelength of from about 7 μm to about 14 μm.

37. The bioreactor system of claim 31, wherein the thermal imaging camera is radiometric.

38. The bioreactor system of claim 31, further comprising:
a vessel having a view port configured to allow the thermal imaging camera to image the exposed surface.

39. The bioreactor system of claim 38, wherein the view port is heated to reduce condensation.

40. The bioreactor system of claim 31, further comprising:
an air curtain configured to reduce vessel condensation to facilitate thermal imaging of the exposed surface.

41. The bioreactor system of claim 40, further comprising a vessel; and
wherein the air curtain is located within the vessel.

42. The bioreactor system of claim 31, wherein the camera is mounted on the support structure so that it is positioned above the vessel and aimed vertically downward so that substantially an entirety of the exposed surface may be imaged.

43. The bioreactor system of claim 38, wherein the vessel is a collapsible bioreactor bag.

44. The bioreactor system of claim 31, wherein the system is configured to identify the presence of and/or a magnitude of foam on the surface of the liquid exposed to the headspace by detecting a rate of temperature change on the exposed surface.

45. The bioreactor system of claim 31, wherein camera and controller identify areas of the exposed surface by comparing a temperature of the exposed surface to a temperature of the liquid to assess whether the exposed surface is a warmer or cooler than the liquid in a predetermined amount that is indicative of foam.

46. The bioreactor system of claim 31, wherein the camera and controller identify areas of the exposed surface as foam by comparing a temperature of the exposed surface to a temperature of the headspace to assess whether the exposed surface is within a predetermined temperature range of the headspace temperature that is indicative of foam.

47. The bioreactor system of claim 31, wherein the headspace is cooler than the liquid in the vessel.
